(19) United States
(12) Patent Application Publication
(10) Pub. No.: US 2010/0048649 A1
(43) Pub. Date: Feb. 25, 2010

(54) COMBINATIONS COMPRISING BICYCLIC S1P LYASE INHIBITORS

(76) Inventor: Tamas Oravecz, The Woodlands, TX (US)

Correspondence Address: LEXICON PHARMACEUTICALS, INC., 8800 TECHNOLOGY FOREST PLACE, THE WOODLANDS, TX, 77381-1160, US

(21) Appl. No.: 12/544,504

(22) Filed: Aug. 20, 2009

Related U.S. Application Data
(60) Provisional application No. 61/090,963, filed on Aug. 22, 2008.

(51) Int. Cl. A61K 31/427; A61K 31/422; A61K 31/4196; A61K 31/4178

(52) U.S. Cl. 514/365; 514/378; 514/383; 514/397

(57) ABSTRACT

Methods and compositions for treating immunological and inflammatory diseases and disorders are disclosed. Particular methods and compositions comprise the administration of an agent that inhibits S1P lyase activity and at least one additional immunosuppressive and/or anti-inflammatory agent.

This application claims priority to U.S. provisional application No. 61/090,963, filed Aug. 22, 2008, the entirety of which is incorporated herein by reference.

### 1. FIELD OF THE INVENTION

Methods and compositions for treating immunological and inflammatory diseases and disorders are disclosed. Particular methods and compositions comprise the administration of an agent that inhibits S1P lyase activity and at least one additional immunosuppressive and/or anti-inflammatory agent.

### 2. BACKGROUND

Sphingosine-1-phosphate (SI P) is a bioactive molecule with potent effects on multiple organ systems. Saba, J. D. and Hla, T. Circ. Res. 94:724-734 (2004). Although some believe the compound is an intracellular secondary messenger, its mode of action is still a subject of debate. Id. Indeed, even its metabolism is poorly understood. Hla, T., Science 309:1682-3 (2005). Researchers currently believe that S1P is formed by the phosphorylation of sphingosine, and degraded by dephosphorylation or cleavage. Its cleavage into ethanolamine phosphate and a long-chain aldehyde is reportedly catalyzed by S1P lyase. Id.; Pyne & Pyne, Biochem J. 349:385-402 (2000). S1P lyase is a vitamin B₆-dependent enzyme localized in the membrane of the endoplasmic reticulum. Van Veldhoven and Mannaerts, J. Biol. Chem. 266:12502-12507 (1991); Van Veldhoven and Mannaerts, Adv. Lipid. Res. 26:69 (1993). The polynucleotide and amino acid sequences of human S1P lyase and its gene products are described in PCT Patent Application No. WO 99/16888.

S1P lyase inhibitors have been recently reported. See, e.g., U.S. patent application Ser. Nos. 11/698,253, filed Jan. 25, 2007, and 12/038,872, filed Feb. 28, 2008.

### 3. SUMMARY OF THE INVENTION

This invention is directed, in part, methods of treating, managing or preventing an immunological or inflammatory disease or disorder, which comprise inhibiting S1P lyase activity in a patient in need thereof and administering to the patient an immunosuppressant and/or an anti-inflammatory agent.

Inhibition of S1P lyase activity can be achieved by administering to the patient a compound of formula I:

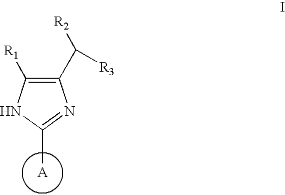

or a pharmaceutically acceptable salt thereof, the substituents of which are defined herein.

This invention also encompasses pharmaceutical compositions comprising compounds of formula I and one or more additional active agents.

### 4. DETAILED DESCRIPTION

This invention results, in part, from discoveries relating to compounds that are believed to inhibit S1P lyase in vivo.

### 4.1. Definitions

Unless otherwise indicated, the term “alkenyl” means a straight chain, branched and/or cyclic hydrocarbon having from 2 to 20 (e.g., 2 to 10 or 2 to 6) carbon atoms, and including at least one carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

Unless otherwise indicated, the term “alkyl” means a straight chain, branched and/or cyclic (“cycloalkyl”) hydrocarbon having from 1 to 20 (e.g., 1 to 10 or 1 to 4) carbon atoms. Alkyl moieties having from 1 to 4 carbons are referred to as “lower alkyl.” Examples of alkyl groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). The term “alkyl” includes saturated hydrocarbons as well as alkenyl and alkynyl moieties.

Unless otherwise indicated, the term “alkylaryl” or “alkyl-aryl” means an alkyl moiety bound to an aryl moiety.

Unless otherwise indicated, the term “alkylheteroaryl” or “alkyl-heteroaryl” means an alkyl moiety bound to a heteroaryl moiety.

Unless otherwise indicated, the term “alkylheterocycle” or “alkyl-heterocycle” means an alkyl moiety bound to a heterocycle moiety.

Unless otherwise indicated, the term “alkynyl” means a straight chain, branched or cyclic hydrocarbon having from 2 to 20 (e.g., 2 to 20 or 2 to 6) carbon atoms, and including at least one carbon-carbon triple bond. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

Unless otherwise indicated, the term “alkoxy” means an —O-alkyl group. Examples of alkoxy groups include —OCH₃, —OCH₂CH₃, —O(CH₂)₂CH₃, —O(CH₂)₃CH₃, —O(CH₂)₄CH₃, —O(cyclopenyl) and —O(CH₂)₅CH₃.

Unless otherwise indicated, the term “aryl” means an aromatic ring or an aromatic or partially aromatic ring system composed of carbon and hydrogen atoms. An aryl moiety may comprise multiple rings bound or fused together. Examples of aryl moieties include, but are not limited to, anthracenyl, azulenyl, biphenyl, fluorenyl, indan, indenyl, naphthyl, phenanthrenyl, phenyl, 1,2,3,4-tetrahydro-naphthalene, and tolyl.

Unless otherwise indicated, the term “arylalkyl” or “aryl-alkyl” means an aryl moiety bound to an alkyl moiety.

Unless otherwise indicated, the term “circulating lymphocyte reduction agent” means a compound that has a CLRF of greater than about 20 percent.

Unless otherwise indicated, the term “circulating lymphocyte reduction factor” or “CLRF” means the decrease in the number of circulating lymphocytes in mice caused by oral administration of a single dose of a compound at 100 mg/kg, as determined by the method described in the Examples, below.

Unless otherwise indicated, the terms “halogen” and “halo” encompass fluorine, chlorine, bromine, and iodine.

Unless otherwise indicated, the term “heteroalkyl” refers to an alkyl moiety (e.g., linear, branched or cyclic) in which at least one of its carbon atoms has been replaced with a heteroatom (e.g., N, O or S).

Unless otherwise indicated, the term “heteroaryl” means an aryl moiety wherein at least one of its carbon atoms has been replaced with a heteroatom (e.g., N, O or S). Examples include, but are not limited to, acridinyl, benzimidazolyl, benzofuranyl, benzoisothiazolyl, benzoisoxazolyl, benzoquinazolinyl, benzothiazolyl, benzoxazolyl, furyl, imidazolyl, indolyl, isothiazolyl, isoxazolyl, oxadiazolyl, oxazolyl, phthalazinyl, pyrazinyl, pyrazolyl, pyridazinyl, pyridyl, pyrimidinyl, pyrimidyl, pyrrolyl, quinazolinyl, quinolinyl, tetrazolyl, thiazolyl, and triazinyl.

Unless otherwise indicated, the term “heteroarylalkyl” or “heteroaryl-alkyl” means a heteroaryl moiety bound to an alkyl moiety.

Unless otherwise indicated, the term “heterocycle” refers to an aromatic, partially aromatic or non-aromatic monocyclic or polycyclic ring or ring system comprised of carbon, hydrogen and at least one heteroatom (e.g., N, O or S). A heterocycle may comprise multiple (i.e., two or more) rings fused or bound together. Heterocycles include heteroaryls. Particular heterocycles are 5- to 13-membered heterocycles containing 1 to 4 heteroatoms selected from nitrogen, oxygen, and sulphur. Others are 5- to 10-membered heterocycles containing 1 to 4 heteroatoms selected from nitrogen, oxygen, and sulphur. Examples of heterocycles include benzo[1,3]dioxolyl, 2,3-dihydro-benzo[1,4]dioxinyl, cinnolinyl, furanyl, hydantoinyl, morpholinyl, oxetanyl, oxiranyl, piperazinyl, piperidinyl, pyrrolidinonyl, pyrrolidinyl, tetrahydrofuranyl, tetrahydropyranyl, tetrahydropyridinyl, tetrahydropyrimidinyl, tetrahydrothiophenyl, tetrahydrothiopyranyl and valerolactamyl.

Unless otherwise indicated, the term “heterocyclealkyl” or “heterocycle-alkyl” refers to a heterocycle moiety bound to an alkyl moiety.

Unless otherwise indicated, the term “heterocycloalkyl” refers to a non-aromatic heterocycle.

Unless otherwise indicated, the term “heterocycloalkylalkyl” or “heterocycloalkyl-alkyl” refers to a heterocycloalkyl moiety bound to an alkyl moiety.

Unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

Unless otherwise indicated, the term “pharmaceutically acceptable salts” refers to salts prepared from pharmaceutically acceptable non-toxic acids or bases including inorganic acids and bases and organic acids and bases. Suitable pharmaceutically acceptable base addition salts include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art. See, e.g., Remington's Pharmaceutical Sciences (18th ed., Mack Publishing, Easton Pa.: 1990) and Remington: The Science and Practice of Pharmacy (19th ed., Mack Publishing, Easton Pa.: 1995).

Unless otherwise indicated, the terms “prevent,” “preventing” and “prevention” contemplate an action that occurs before a patient begins to suffer from the specified disease or disorder, which inhibits or reduces the severity of the disease or disorder. In other words, the terms encompass prophylaxis.

Unless otherwise indicated, a “prophylactically effective amount” of a compound is an amount sufficient to prevent a disease or condition, or one or more symptoms associated with the disease or condition, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

Unless otherwise indicated, the term “S1P level enhancing agent” means a compound that has a SLEF of at least about 10-fold.

Unless otherwise indicated, the term “S1P level enhancing factor” or “SLEF” means the increase in S1P in the spleens of mice caused by oral administration of a single dose of a compound at 100 mg/kg, as determined by the method described in the Examples, below.

Unless otherwise indicated, the term “stereoisomeric mixture” encompasses racemic mixtures as well as stereomerically enriched mixtures (e.g., R/S=30/70, 35/65, 40/60, 45/55, 55/45, 60/40, 65/35 and 70/30).

Unless otherwise indicated, the term “stereomerically pure” means a composition that comprises one stereoisomer of a compound and is substantially free of other stereoisomers of that compound. For example, a stereomerically pure composition of a compound having one stereocenter will be substantially free of the opposite stereoisomer of the compound. A stereomerically pure composition of a compound having two stereocenters will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound.

Unless otherwise indicated, the term “substituted,” when used to describe a chemical structure or moiety, refers to a derivative of that structure or moiety wherein one or more of its hydrogen atoms is substituted with a chemical moiety or functional group such as, but not limited to, alcohol, aldehylde, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amidinyl (—C(NH)NH-alkyl or —C(NR)NH₂), amine (primary, secondary and tertiary such as alkylamino, arylamino, arylalkylamino), aroyl, aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carbonyl, carboxyl, carboxylic acid, carboxylic acid anhydride, carboxylic acid chloride, cyano, ester, epoxide, ether (e.g., methoxy, ethoxy), guanidino, halo, haloalkyl (e.g., —CCl₃, —CF₃, —C(CF₃)₃), heteroalkyl, hemiacetal, imine (primary and secondary), isocyanate, isothiocyanate, ketone, nitrile, nitro, oxo, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) and urea (—NHCONH-alkyl-).

Unless otherwise indicated, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder, or retards or slows the progression of the disease or disorder.

Unless otherwise indicated, the term “include” has the same meaning as “include, but are not limited to,” and the term “includes” has the same meaning as “includes, but is not limited to.” Similarly, the term “such as” has the same meaning as the term “such as, but not limited to.”

Unless otherwise indicated, one or more adjectives immediately preceding a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

It should be noted that a chemical moiety that forms part of a larger compound may be described herein using a name commonly accorded it when it exists as a single molecule or a name commonly accorded its radical. For example, the terms “pyridine” and “pyridyl” are accorded the same meaning when used to describe a moiety attached to other chemical moieties. Thus, the two phrases “XOH, wherein X is pyridyl” and “XOH, wherein X is pyridine” are accorded the same meaning, and encompass the compounds pyridin-2-ol, pyridin-3-ol and pyridin-4-ol.

It should also be noted that if the stereochemistry of a structure or a portion of a structure is not indicated with, for example, bold or dashed lines, the structure or the portion of the structure is to be interpreted as encompassing all stereoisomers of it. Moreover, any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit.

### 4.2. Compounds

This invention relates to methods of using, and compositions comprising, a compound that decreases S1P lyase activity in vivo and at least one additional pharmacological agent that affects immune or inflammatory response.

4.2.1. S1P Lyase Inhibitors

This invention contemplates the use of S1P lyase inhibitors disclosed in U.S. patent application Ser. No. 12/038,872, filed Feb. 28, 2008 (publication no. US-2009-0030050-A1). Particular compounds are of formula I:

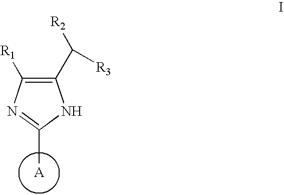

or are pharmaceutically acceptable salts thereof, wherein: A is an optionally substituted heterocycle; R₁ is OR_{1A}, OC(O)R_{1A}, C(O)OR_{1A}, hydrogen, halogen, nitrile, or optionally substituted alkyl, aryl, alkylaryl, arylalkyl, heteroalkyl, heterocycle, alkylheterocycle, or heterocyclealkyl; R₂ is OR_{2A}, OC(O)R_{2A}, hydrogen, halogen, or optionally substituted alkyl, aryl, alkylaryl, arylalkyl, heteroalkyl, heterocycle, alkylheterocycle, or heterocyclealkyl; R₃ is N(R_{3A})₂, hydrogen, hydroxy, or optionally substituted alkyl, aryl, alkylaryl, arylalkyl, heteroalkyl, heterocycle, alkylheterocycle, or heterocyclealkyl; and each of R_{1A}, R_{2A}, and R_{3A} is independently hydrogen or optionally substituted alkyl, aryl, alkylaryl, arylalkyl, heteroalkyl, heterocycle, alkylheterocycle, or heterocyclealkyl.

Particular compounds are of formula I(a) or I(b):

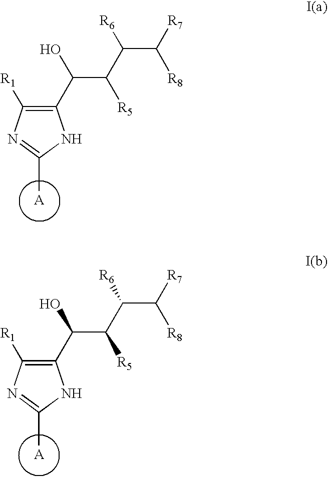

wherein: R₅ is OR_{5A}, OC(O)R_{5A}, N(R_{5B})₂, NHC(O)R_{5B}, hydrogen, or halogen; R₆ is OR_{6A}, OC(O)R_{6A}, N(R_{6B})₂, NHC(O)R_{6B}, hydrogen, or halogen; R₇ is OR_{7A}, OC(O)R_{7A}, N(R_{7B})₂, NHC(O)R_{7B}, hydrogen, or halogen; R₈ is CH₂OR_{8A}, CH₂OC(O)R_{8A}, N(R_{8B})₂, NHC(O)R_{8B}, hydrogen, or halogen; each of R_{1A}, R_{5A}, R_{6A}, R_{7A}, and R_{8A} is independently hydrogen or optionally substituted alkyl, aryl, alkylaryl, arylalkyl, heteroalkyl, heterocycle, alkylheterocycle, or heterocyclealkyl; and each of R_{5B}, R_{6B}, R_{7B} and R_{8B} is independently hydrogen or alkyl optionally substituted with one or more hydroxy or halogen groups.

Some compounds are of formula II:

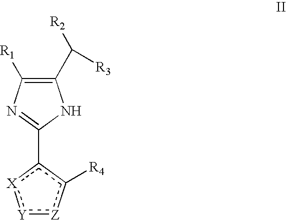

wherein: X is CR₄, CHR₄, N, NR₉, O or S; Y is CR₄, CHR₄, N, NR₉, O or S; Z is CR₄, CHR₄, N, NR₉, O or S; R₁ is OR_{1A}, C(O)OR_{1A}, hydrogen, halogen, nitrile, or optionally substituted alkyl, aryl, alkylaryl, arylalkyl, heteroalkyl, heterocycle, alkylheterocycle, or heterocyclealkyl; R₂ is OR_{2A}, OC(O)R_{2A}, hydrogen, halogen, or optionally substituted alkyl, aryl, alkylaryl, arylalkyl, heteroalkyl, heterocycle, alkylheterocycle, or heterocyclealkyl; R₃ is N(R_{3A})₂, hydrogen, hydroxy, or optionally substituted alkyl, aryl, alkylaryl, arylalkyl, heteroalkyl, heterocycle, alkylheterocycle, or heterocyclealkyl; each of R_{1A}, R_{2A}, and R_{3A} is independently hydrogen or optionally substituted alkyl, aryl, alkylaryl, arylalkyl, heteroalkyl, heterocycle, alkylheterocycle, or heterocyclealkyl; each R₄ is independently OR_{4A}, OC(O)R_{4A}, hydrogen, halogen, or optionally substituted alkyl, aryl, alkylaryl, arylalkyl, heteroalkyl, heterocycle, alkylheterocycle, or heterocyclealkyl; each R₉ is independently hydrogen or optionally substituted alkyl, aryl, alkylaryl, arylalkyl, heteroalkyl, heterocycle, alkylheterocycle, or heterocyclealkyl; and each of R_{1A}, R_{2A}, R_{3A} and R_{4A} is independently hydrogen or optionally substituted alkyl, aryl, alkylaryl, arylalkyl, heteroalkyl, heterocycle, alkylheterocycle, or heterocyclealkyl.

Particular compounds are of formulae II(a) or II(b):

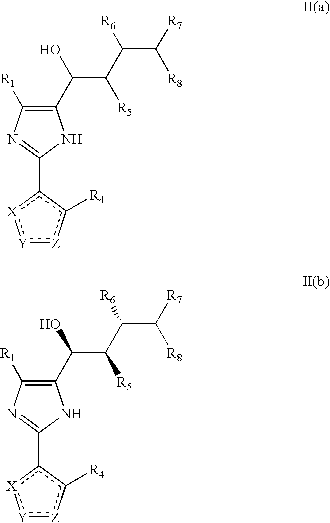

wherein: R₅ is OR_{5A}, OC(O)R_{5A}, N(R_{5B})₂, NHC(O)R_{5B}, hydrogen, or halogen; R₆ is OR_{6A}, OC(O)R_{6A}, N(R_{6B})₂, NHC(O)R_{6B}, hydrogen, or halogen; R₇ is OR_{7A}, OC(O)R_{7A}, N(R_{7B})₂, NHC(O)R_{7B}, hydrogen, or halogen; R₈ is CH₂OR_{8A}, CH₂OC(O)R_{8A}, N(R_{8B})₂, NHC(O)R_{8B}, hydrogen, or halogen; each of R_{1A}, R_{5A}, R_{6A}, R_{7A}, and R_{8A} is independently hydrogen or optionally substituted alkyl, aryl, alkylaryl, arylalkyl, heteroalkyl, heterocycle, alkylheterocycle, or heterocyclealkyl; and each of R_{5B}, R_{6B}, R_{7B} and R_{8B} is independently hydrogen or alkyl optionally substituted with one or more hydroxy or halogen groups.

Some compounds are of formula III:

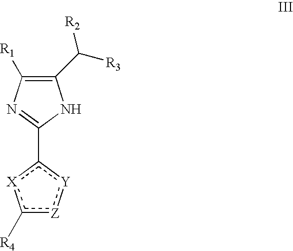

wherein: X is CR₄, CHR₄, N, NR₉, O or S; Y is CR₄, CHR₄, N, NR₉, O or S; Z is CR₄, CHR₄, N, NR₉, O or S; R₁ is OR_{1A}, C(O)OR_{1A}, hydrogen, halogen, nitrile, or optionally substituted alkyl, aryl, alkylaryl, arylalkyl, heteroalkyl, heterocycle, alkylheterocycle, or heterocyclealkyl; R₂ is OR_{2A}, OC(O)R_{2A}, hydrogen, halogen, or optionally substituted alkyl, aryl, alkylaryl, arylalkyl, heteroalkyl, heterocycle, alkylheterocycle, or heterocyclealkyl; R₃ is N(R_{3A})₂, hydrogen, hydroxy, or optionally substituted alkyl, aryl, alkylaryl, arylalkyl, heteroalkyl, heterocycle, alkylheterocycle, or heterocyclealkyl; each of R_{1A}, R_{2A}, and R_{3A} is independently hydrogen or optionally substituted alkyl, aryl, alkylaryl, arylalkyl, heteroalkyl, heterocycle, alkylheterocycle, or heterocyclealkyl; each R₄ is independently OR_{4A}, OC(O)R_{4A}, hydrogen, halogen, or optionally substituted alkyl, aryl, alkylaryl, arylalkyl, heteroalkyl, heterocycle, alkylheterocycle, or heterocyclealkyl; each R₉ is independently hydrogen or optionally substituted alkyl, aryl, alkylaryl, arylalkyl, heteroalkyl, heterocycle, alkylheterocycle, or heterocyclealkyl; and each of R_{1A}, R_{2A}, R_{3A} and R_{4A} is independently hydrogen or optionally substituted alkyl, aryl, alkylaryl, arylalkyl, heteroalkyl, heterocycle, alkylheterocycle, or heterocyclealkyl.

Particular compounds are of formulae III(a) or III(b):

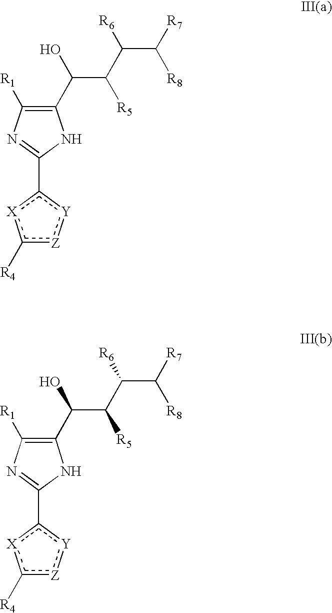

wherein: R₅ is OR_{5A}, OC(O)R_{5A}, N(R_{5B})₂, NHC(O)R_{5B}, hydrogen, or halogen; R₆ is OR_{6A}, OC(O)R_{6A}, N(R_{6B})₂, NHC(O)R_{6B}, hydrogen, or halogen; R₇ is OR_{7A}, OC(O)R_{7A}, N(R_{7B})₂, NHC(O)R_{7B}, hydrogen, or halogen; R₈ is CH₂OR_{8A}, CH₂OC(O)R_{8A}, N(R_{8B})₂, NHC(O)R_{8B}, hydrogen, or halogen; each of R_{1A}, R_{5A}, R_{6A}, R_{7A}, and R_{8A} is independently hydrogen or optionally substituted alkyl, aryl, alkylaryl, arylalkyl, heteroalkyl, heterocycle, alkylheterocycle, or heterocyclealkyl; and each of R_{5B}, R_{6B}, R_{7B} and R_{8B} is independently hydrogen or alkyl optionally substituted with one or more hydroxy or halogen groups.

Referring to the various formulae disclosed herein (e.g., formulae I, II and III), as applicable, in some compounds of the invention, A is a 5-membered optionally substituted heterocycle. Examples include optionally substituted dihydro-imidazole, dihydro-isoxazole, dihydro-pyrazole, dihydro-thiazole, dioxolane, dithiolane, dithiole, imidazole, isoxazole, isoxazolidine, oxathiolane, and pyrazole. In one embodiment, A is not optionally substituted furan, thiophene or pyrrole.

In some compounds, A is a 6-membered optionally substituted heterocycle (e.g., pyrimidine).

In some, X is CR₄ or CHR₄. In some, X is N or NR₉. In some, X is O or S.

In some, Y is CR₄ or CHR₄. In some, Y is N or NR₉. In some, Y is O or S.

In some, Z is CR₄ or CHR₄. In some, Z is N or NR₉. In some, Z is O or S.

In some, X is N and Y is O. In some, X is N and Y is NR₉. In some, X is N and Y is S. In some, X is N and Z is O. In some, X is N and Z is NR₉. In some, X is N and Z is S. In some, X is N, Y is N, and Z is NR₉.

In some, R₁ is hydrogen. In some, R₁ is nitrile. In some, R₁ is optionally substituted lower alkyl. In some, R₁ is OR_{1A} or C(O)OR_{1A} and R_{1A} is, for example, hydrogen or optionally substituted lower alkyl.

In some, R₂ is OR₂A. In some, R₂ is OC(O)R_{2A} and R_{2A} is, for example, hydrogen. In some, R₂ is halogen.

In some, R₃ is optionally substituted alkyl (e.g., alkyl substituted with one or more halogen or OR_{3A} moieties, wherein R_{3A} is, for example, hydrogen or acetate). In some, R₃ is hydrogen. In some, R₃ is hydroxyl. In some, R₃ is optionally substituted heteroalkyl (e.g., alkoxy). In some, R₃ is heteroalkyl substituted with one or more halogen, hydroxyl or acetate.

In some, R₄ is hydrogen or optionally substituted alkyl, aryl or alkylaryl.

In some, each of R₅, R₆, R₇, and R₈ is hydrogen or halogen. In some, one or more of R₅, R₆, R₇, and R₈ is hydroxyl or acetate. In some, all of R₅, R₆, R₇, and R₈ are hydroxyl.

In some, R₉ is hydrogen or optionally substituted alkyl, aryl or alkylaryl.

Compounds of the invention may contain one or more stereocenters, and can exist as racemic mixtures of enantiomers or mixtures of diastereomers. This invention encompasses stereomerically pure forms of such compounds, as well as mixtures of those forms. Stereoisomers may be asymmetrically synthesized or resolved using standard techniques such as chiral columns or chiral resolving agents. See, e.g., Jacques, J., et al, Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGraw Hill, NY, 1962); and Wilen, S. H., Tables of Resolving Agents and Optical Resolutions, p. 268 (E. L. Eliel, Ed., Univ. of Notre Dame Press, Notre Dame, 1N, 1972).

This invention further encompasses stereoisomeric mixtures of compounds disclosed herein. It also encompasses configurational isomers of compounds disclosed herein, either in admixture or in pure or substantially pure form, such as cis (Z) and trans (E) alkene isomers and syn and anti oxime isomers.

Particular compounds of the invention are circulating lymphocyte reduction agents. Certain compounds inhibit the amount of circulating lymphocytes, as determined using the method described in the Examples, by greater than about 20, 50, 75, 100, 150 or 200 percent.

Without being limited by theory, compounds of the invention are believed to affect the S1P metabolic pathway, and may inhibit S1P lyase directly or indirectly in vivo. Particular compounds are S1P level enhancing agents. Certain compounds increase the amount of S1P, as determined using the method described below in the Examples, by greater than about 10, 15, 20, 25, or 30-fold.

Compounds of the invention can be prepared by methods known in the art (e.g., by varying and adding to the approaches described in Pyne, S. G., ACGC Chem. Res. Comm. 11:108-112 (2000); Halweg, K. M. and Büchi, G., J. Org. Chem. 50:1134-1136 (1985)). Compounds can also be made by the methods disclosed below and variants thereof, which will be apparent to those of ordinary skill in the art.

For example, compounds of formula I can be prepared from commercially available, and/or readily prepared nitriles, as shown below:

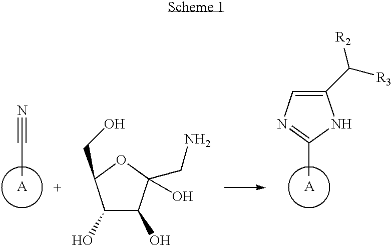

wherein, for example, the reactants are combined with one equivalent of NaOMe in MeOH at room temperature, followed by the addition of acid (e.g., aqueous HCl).

Compounds of the invention (i.e., compounds disclosed herein) may contain one or more stereocenters, and can exist as racemic mixtures of enantiomers or mixtures of diastereomers. This invention encompasses stereomerically pure forms of such compounds, as well as mixtures of those forms. Stereoisomers may be asymmetrically synthesized or resolved using standard techniques such as chiral columns or chiral resolving agents. See, e.g., Jacques, J., et al, Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGraw Hill, NY, 1962); and Wilen, S. H., Tables of Resolving Agents and Optical Resolutions, p. 268 (E. L. Eliel, Ed., Univ. of Notre Dame Press, Notre Dame, Ind., 1972).

This invention further encompasses stereoisomeric mixtures of compounds disclosed herein. It also encompasses configurational isomers of compounds disclosed herein, either in admixture or in pure or substantially pure form, such as cis (Z) and trans (E) alkene isomers and syn and anti oxime isomers.

Certain compounds are circulating lymphocyte reduction agents. Particular compounds inhibit the amount of circulating lymphocytes, as determined using the method described in the Examples, by greater than about 20, 50, 75, 100, 150 or 200 percent.

Certain compounds inhibit S1P lyase directly or indirectly in vivo, and are S1P level enhancing agents. Particular compounds increase the amount of S1P, as determined using the method described below in the Examples, by greater than about 10, 15, 20, 25, or 30-fold.

4.2.2. Immunosuppressive and Anti-Inflammatory Agents

Immunosuppressants suitable for use in the methods and compositions of this invention include those known in the art. Examples include aminopterin, azathioprine, cyclosporin A, D-penicillamine, gold salts, hydroxychloroquine, leflunomide, methotrexate, minocycline, rapamycin, sulfasalazine, tacrolimus (FK506), and pharmaceutically acceptable salts thereof. A particular immunosuppressant is methotrexate.

Additional examples include anti-TNF antibodies, such as adalimumab, certolizumab pegol, etanercept, and infliximab. Others include interleukin-1 blockers, such as anakinra. Others include anti-B cell (CD20) antibodies, such as rituximab. Others include T cell activation blockers, such as abatacept.

Additional examples include inosine monophosphate dehydrogenase inhibitors, such as mycophenolate mofetil (CellCept®) and mycophenolic acid (Myfortic®).

Anti-inflammatory drugs suitable for use in the methods and compositions of this invention include those known in the art. Examples include glucocorticoids and NSAIDs.

Examples of glucocorticoids include aldosterone, beclometasone, betamethasone, cortisone, deoxycorticosterone, dexamethasone, fluorocortisones, hydrocortisone, methylprednisolone, prednisolone, prednisone, triamcinolone, and pharmaceutically acceptable salts thereof.

Examples of NSAID include salicylates (e.g., aspirin, amoxiprin, benorilate, choline magnesium salicylate, diflunisal, faislamine, methyl salicylate, magnesium salicylate, salicyl salicylate, and pharmaceutically acceptable salts thereof), arylalkanoic acids (e.g., diclofenac, aceclofenac, acemetacin, bromfenac, etodolac, indometacin, nabumetone, sulindac, tolmetin, and pharmaceutically acceptable salts thereof), arylpropionic acids (e.g., ibuprofen, carprofen, fenbufen, fenoprofen, flurbiprofen, ketoprofen, ketorolac, loxoprofen, naproxen, oxaprozin, tiaprofenic acid, suprofen, and pharmaceutically acceptable salts thereof), arylanthranilic acids (e.g., meclofenamic acid, mefenamic acid, and pharmaceutically acceptable salts thereof), pyrazolidine derivatives (e.g., azapropazone, metamizole, oxyphenbutazone, phenylbutazone, sulfinprazone, and pharmaceutically acceptable salts thereof), oxicams (e.g., lornoxicam, meloxicam, piroxicam, tenoxicam, and pharmaceutically acceptable salts thereof), COX-2 inhibitors (e.g., celecoxib, etoricoxib, lumiracoxib, parecoxib, rofecoxib, valdecoxib, and pharmaceutically acceptable salts thereof), and sulphonanilides (e.g., nimesulide and pharmaceutically acceptable salts thereof).

### 4.3. Methods of Use

This invention encompasses a method of treating, managing or preventing an immunological or inflammatory disease or disorder in a patient (e.g., a human), which comprises inhibiting S1P lyase activity in the patient and administering to the patient an immunosuppressive and/or anti-inflammatory drug that acts by a different mechanism.

Also encompassed by the invention is a method of reducing the dose of an immunosuppressive and/or anti-inflammatory drug necessary to treat, manage or prevent an immunological or inflammatory disease or disorder, which comprises adjunctively administering to the patient a compound that inhibits S1P lyase activity. This method allows one to reduce toxicities associated with many immunosuppressive and anti-inflammatory drugs while maintaining their efficacy.

Examples of immunological and inflammatory diseases and disorder include Addison's Disease, anti-phospholipid syndrome, asthma, atopic dermatitis, autoimmune atrophic gastritis, achlorhydra autoimmune, Behcet's disease, Celiac Disease, chronic idiopathic urticaria, Chronic infantile neurological cutaneous and articular (CINCA) syndrome (also known as neonatal-onset multisystem inflammatory disease (NOMID)), chronic obstructive pulmonary disease (COPD), Crohn's Disease, Cushing's Syndrome, dermatomyositis, Goodpasture's Syndrome, graft-vs-host disease, Grave's Disease, Hashimoto's thyroiditis, idiopathic adrenal atrophy, idiopathic thrombocytopenia, Lambert-Eaton Syndrome, lupus erythematosus, multiple sclerosis, pemphigoid, pemphigus vulgaris, pernicious anemia, pollinosis, polyarteritis nodosa, primary biliary cirrhosis, primary sclerosing cholangitis, psoriasis, Raynauds, Reiter's Syndrome, relapsing polychondritis, rheumatoid arthritis, rhinitis, Schmidt's Syndrome, sepsis, Sjogren's Syndrome, sympathetic ophthalmia, Takayasu's Arteritis, temporal arteritis, thyrotoxicosis, transplant rejection (e.g., tissue transplantation, bone marrow transplantation), ulcerative colitis, uveitis, vasculitis and Wegener's granulomatosis.

The amount, route of administration and dosing schedule of a compound will depend upon factors such as the specific indication to be treated, prevented, or managed, and the age, sex and condition of the patient. The roles played by such factors are well known in the art, and may be accommodated by routine experimentation. In a particular embodiment, a compound of formula I is administered to a human patient in an amount of about 0.5, 1, 2.5 or 5 mpk.

### 4.4. Pharmaceutical Formulations

This invention encompasses pharmaceutical compositions comprising at least two active pharmacological ingredients. Certain pharmaceutical compositions are single unit dosage forms suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, oral administration requires enteric coatings to protect the compounds of this invention from degradation within the gastrointestinal tract. Similarly, a formulation may contain ingredients that facilitate delivery of the active ingredient(s) to the site of action. For example, compounds may be administered in liposomal formulations, in order to protect them from degradative enzymes, facilitate transport in circulatory system, and effect delivery across cell membranes to intracellular sites.

The composition, shape, and type of a dosage form will vary depending on its use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18^{th} ed. (Mack Publishing, Easton Pa.: 1990).

4.4.1. Oral Dosage Forms

Pharmaceutical compositions of the invention suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See, e.g., Remington's Pharmaceutical Sciences, 18^{th} ed. (Mack Publishing, Easton Pa.: 1990).

Typical oral dosage forms are prepared by combining the active ingredient(s) in an intimate admixture with at least one excipient according to conventional pharmaceutical compounding techniques. Excipients can take a wide variety of forms depending on the form of preparation desired for administration.

Because of their ease of administration, tablets and capsules represent the most advantageous oral dosage unit forms. If desired, tablets can be coated by standard aqueous or nonaqueous techniques. Such dosage forms can be prepared by conventional methods of pharmacy. In general, pharmaceutical compositions and dosage forms are prepared by uniformly and intimately admixing the active ingredients with liquid carriers, finely divided solid carriers, or both, and then shaping the product into the desired presentation if necessary. Disintegrants may be incorporated in solid dosage forms to facility rapid dissolution. Lubricants may also be incorporated to facilitate the manufacture of dosage forms (e.g., tablets).

4.4.2. Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are specifically sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

4.4.3. Transdermal, Topical and Mucosal Dosage Forms

Transdermal, topical, and mucosal dosage forms include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 18^{th} ed. (Mack Publishing, Easton Pa.: 1990); and Introduction to Pharmaceutical Dosage Forms, 4^{th} ed. (Lea & Febiger, Philadelphia: 1985). Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms are well known to those skilled in the pharmaceutical arts, and depend on the particular tissue to which a given pharmaceutical composition or dosage form will be applied.

Depending on the specific tissue to be treated, additional components may be used prior to, in conjunction with, or subsequent to treatment with active ingredients of the invention. For example, penetration enhancers may be used to assist in delivering active ingredients to the tissue.

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates may also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different forms of the active ingredients can be used to further adjust the properties of the resulting composition.

### 5. EXAMPLES

Aspects of this invention can be understood from the following examples, which do not limit its scope.

### 5.1. Synthesis of (1R,2S,3R)-1-(2-(5-methylisoxazol-3-yl)-1H-imidazol-5-yl)butane-1,2,3,4-tetraol

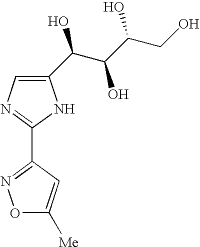

The captioned compound was prepared by General Method A, which is shown below in Scheme 2:

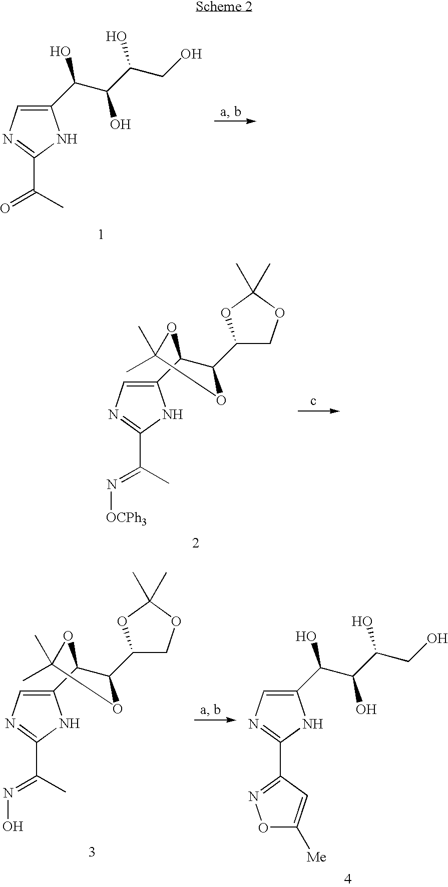

wherein: a is DCE:(MeO)₂CMe₂ (1:1), p-TsOH, 70° C.; b is Ph₃CONH₂, MeOH, 1N HCl (1.0 equiv.); c is 2 N HCl/dioxane; d is n-BuLi 4.0 equiv, THF, 0° C., then N-methyl-N-methoxyacetamide 5.0 equiv.; and e is 1N HCl:dioxane (1:1).

In particular, to a slurry of 1 (4.34 g, 18.87 mmol) in dichloromethane (30 ml) was added 2,2-dimethoxypropane (30 ml) followed by p-toluenesulfonic acid monohydrate (900 mgs, 4.72 mmol). The slurry was heated to 70° C. for 16 h, then cooled to room temperature, and treated with excess triethylamine (1 ml). The reaction was concentrated and dried by toluene azeotrope to give an amber solid that was carried on immediately without purification.

The amber solid was dissolved in MeOH (100 ml), and then treated with N-trityl hydroxylamine (6.75 g, 24.53 mmol) and 1N HCl (18.5 ml, 18.5 mmol). The reaction became clear after 1 h, and was maintained at room temperature for 18 h. At completion, the reaction was neutralized to pH=7 with 10N NaOH solution, then concentrated under reduced pressure. The crude material was purified by chromatography on silica gel (32-63 μm, 10% MeOH:CH₂Cl₂ w/1% NH₄OH) to provide the protected product 2 (9.8 g, 91% yield, 2 steps) as a white foam.

Anhydrous 4M dioxane (20 ml) was added to a solution of 2 (3.11 g, 5.48 mmol) in anhydrous dioxane (40 ml). After 1 h, the reaction was concentrated under vacuum, then redissolved in anhydrous DCM (60 ml), treated with excess triethylamine (5 ml), then concentrated again. The crude product was flashed over silica gel (3-8% MeOH:CH₂Cl₂ w/0.5-1.0% NH₄OH) to provide the oxime 3 (1.05 g, 59% yield) as a white foam.

To a −45° C. solution of 3 (500 mgs, 1.54 mmol) in THF (15 ml) was added dropwise a 1.6 M hexane solution of n-BuLi (3.85 ml, 6.16 mmol). After 10 min, N-methyl-N-methoxyacetamide (0.79 ml, 7.69 mmol) was added dropwise and the reaction was allowed to warm to room temperature. After 2 h, the reaction was quenched by addition of NH₄Cl (10 ml) and diluted with water (5 ml) to dissolve solids. The layers were separated and the aqueous layer was extracted with Et₂O (2×20 ml). The combined organics were washed with brine (25 ml), then dried over MgSO₄ and concentrated under vacuum. The resulting foam was purified by flash chromatography over silica gel (60-90% EtOAc:hexane) to provide a white foam solid.

To a solution of this intermediate white solid in dioxane (5 ml) was added 1N HCl (5 ml). The reaction was heated to 80° C. for 2 h, and then concentrated under reduced pressure to dryness. The resulting glassy solid was lyophilized from water (8 ml) to provide 4 (224 mgs, 48% yield, 2 steps) as a fluffy white powder. MS m/z C₁₁H₁₅N₃O₅ [M+H]⁺=270; ¹H NMR (400 MHz, D₂O): δ 7.54 (s, 1H), 6.7 (s, 1H), 5.2 (s, 1H), 3.83-3.59 (m, 4H), 2.49 (s, 1H); ¹³C NMR (100 MHz, D₂O): δ 174.3, 150.0, 136.6, 135.0, 118.1, 101.0, 73.1, 71.0, 65.0, 63.2.

### 5.2. Synthesis of (1R,2S,3R)-1-(2-(5-ethylisoxazol-3-yl)-1H-imidazol-5-yl)butane-1,2,3,4-tetraol

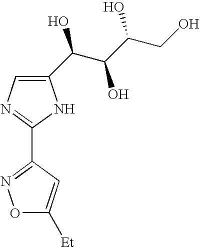

This compound was synthesized by General Method A, by alkylating intermediate 3 with N-methyl-N-methoxy ethyl amide. MS m/z C₁₂H₁₇N₃O₅ [M+H]⁺=284; ¹H NMR (400 MHz, D₂O): δ 7.24 (s, 1H), 6.54 (s, 1H), 4.95 (s, 1H), 3.84-3.56 (m, 4H), 2.82-2.77 (m, 2H), 1.25 (t, J=6.0 Hz, 3H).

### 5.3. Synthesis of (1R,2S,3R)-1-(2-(isoxazol-3-yl)-1H-imidazol-5-yl)butane-1,2,3,4-tetraol

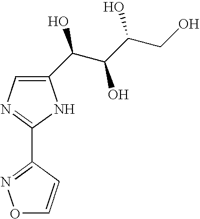

This compound was prepared by modifying General Method A as shown below in Scheme 3:

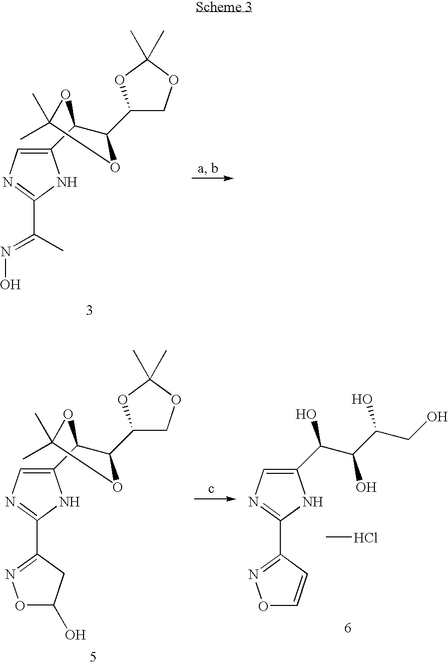

wherein: a is n-BuLi (4.0 equiv), THF, 0° C., then DMF (5.0 equiv.); b is TFAA, pyridine, DCM; and c is 1N HCl:dioxane (1:1), 50° C.

In particular, to a −45° C. solution of 3 (424 mgs, 1.30 mmol) in THF (15 ml) was added dropwise a 2.5 M hexane solution of n-BuLi (2.1 ml, 5.25 mmol). After 10 min, anhydrous DMF (0.505 ml, 6.52 mmol) was added dropwise and the reaction was allowed to warm to room temperature. After 2 h, the reaction was quenched by addition of NH₄Cl (10 ml) and diluted with water (5 ml) to dissolve solids. The layers were separated and the aqueous layer was washed with Et₂O (2×20 ml). The combined organics were washed with brine (25 ml), then dried over MgSO₄ and concentrated under vacuum. The resulting foam was flashed over silica gel (3-6% MeOH: CH₂Cl₂ with 0.5% NH₄OH) to provide the hemiacetal 5 (220 mgs, 47% yield) as a white foam.

To a 0° C. solution of 5 (130 mgs, 0.37 mmol) in THF was sequentially added pyridine (120 μl, 1.48 mmol) and trifluoroacetic acid anhydride. The reaction was warmed to room temperature for 10 min, and then heated to 55° C. for 16 h. At completion, the reaction was concentrated under vacuum, then purified by flash chromatography over silica gel (60-90% EtOAc:hexane) to provide the heterobicycle bisketal (60 mgs, 47% yield) as a white foam that was finally deprotected using standard acidic conditions to give Example 3 compound as a white crystalline solid. MS m/z C₁₀H₁₃N₃O₅ [M+H]⁺=256; ¹H NMR (400 MHz, D₂O) δ 8.87 (s, 1H), 7.55 (s, 1H), 7.05 (s, 1H), 5.21 (s, 1H), 3.75 (m, 3H), 3.63 (m, 2H).

### 5.4. Alternate Synthesis of (1R,2S,3R)-1-(2-(isoxazol-3-yl)-1H-imidazol-5-yl)butane-1,2,3,4-tetraol

The captioned compound was also prepared by the approach referred to herein as General Method B, which is shown below in Scheme 4:

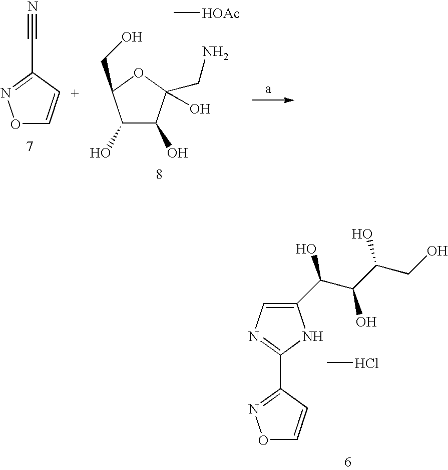

wherein: a is 1.0 equiv NaOMe in MeOH, at room temperature, then aq. HCl.

In particular, to a room temperature solution of the nitrile 7 (600 mgs, 6.38 mmol) in MeOH (10 ml) was added 25% w/v MeONa (0.83 ml, 3.83 mmol). After 3 h, fructosamine-acetate (1.53 g, 6.38 mmol) was added and the solution was maintained at room temperature with vigorous stirring for 5 h. Another portion of 25% w/v MeONa (0.66 ml, 3.19 mmol) was then added to the thick slurry. After 16 h, the reaction was filtered and the cake washed with cold MeOH. The cake was then treated with 1N HCl (20 ml) and filtered. The aqueous solution was concentrated under vacuum to constant weight to provide title compound (1.30 g, 66% yield) as a white powder.

### 5.5. Synthesis of (1R,2S,3R)-1-(2-(2-methylthiazol-4-yl)-1H-imidazol-4-yl)butane-1,2,3,4-tetraol

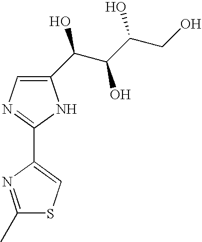

The title compound was prepared by General Method B using 2-methylthiazole-4-carbonitrile (1.023 g, 8.25 mmol), sodium methoxide in methanol (25 wt %, 1.07 ml, 4.95 mmol), methanol (8.25 ml) and compound 8 (2.00 g, 8.26 mmol). After 2.5 days, and additional portion of sodium methoxide in methanol (25 wt %, 0.891 ml, 4.125 mmol) was added. After 24 hours, the solid that had formed was collected by filtration and washed with cold methanol to afford the title compound (1.70 g, 5.96 mmol, 72% yield). MS m/z C₁₁H₁₅N₃O₄S [M+H]=286; ¹H NMR (400 MHz, CD₃OD) δ 2.81 (s, 3H), 3.67-3.75 (m, 2H), 3.77-3.88 (m, 2H), 5.21 (s, 1H), 7.47 (s, 1H), 8.35 (s, 1H).

### 5.6. Synthesis of (1R,2S,3R)-1-(2-(1-benzyl-1H-1,2,4-triazol-3-yl)-1H-imidazol-4-yl)butane-1,2,3,4-tetraol hydrochloride

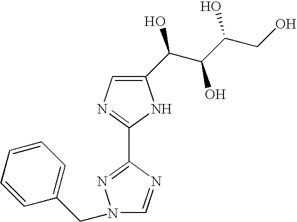

The captioned compound was prepared by General Method B with the following alterations: 1-benzyl-1H-1,2,4-triazole-3-carbonitrile (2.10 g, 11.4 mmol) was dissolved in methanol (12 ml) and treated with sodium methoxide in methanol (25 wt %, 1.48 ml, 6.8 mmol) and stirred for 18 h and 8 was added and the reaction stirred for 18 h. The resulting solid was isolated by filtration, washed with methanol and dried in vacuo to afford a white solid (3.20 g, 9.28 mmol, 81% yield). This solid was suspended in THF (50 ml), cooled in an ice bath and HCl (4 M in dioxane, 7.5 ml, 30 mmol) was added. The ice bath was removed and the suspension was stirred for 4 h. The solid was isolated by filtration, washed with THF and dried in vacuo to afford the title compound (3.50 g, 9.19 mmol, 99% yield) as a white solid. MS m/z C₁₆H₁₉N₅O₄ [M+H]+=346; ¹H NMR (400 MHz, CD₃OD) δ 2.81 (s, 3H), 3.67-3.75 (m, 2H), 3.77-3.88 (m, 2H), 5.21 (s, 1H), 7.47 (s, 1H), 8.35 (s, 1H).

### 5.7. Synthesis of (1R,2S,3R)-1-(1H,1′H-2,2′-biimidazol-5-yl)butane-1,2,3,4-tetraol

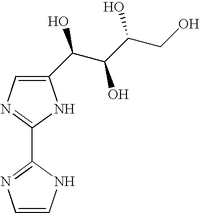

The captioned compound was prepared by General Method B with the following alterations. To a solution of 1H-imidazole-2-carbonitrile (0.39 g, 4.17 mmol) in methanol (4.8 ml) was added a solution of sodium methoxide in methanol (25 wt %, 0.54 g, 0.57 ml, 2.50 mmol), stirred for 16 h and compound 8 (0.964 g, 4.17 mmol) was added in 10 ml of MeOH. A precipitate formed and was filtered and washed with acetone (15 ml). The filtrate was concentrated to dryness, and was purified by preparative HPLC (10 mM aq ammonium acetate/acetonitrile) to give the title compound (0.0141 g, 0.0554 mmol) as an off-white solid. MS m/z C₁₀H₁₄N₄O₄ [M+H]⁺=255; ¹H NMR (400 MHz, CD₃OD) δ 3.56-3.57 (m, 2H), 3.67-3.74 (m, 2H), 4.90 (s, 1H), 7.04 (s, 1H).

### 5.8. Synthesis of (1R,2S,3R)-1-(2-(5-methoxy-4,5-dihydroisoxazol-3-yl)-1H-imidazol-5-yl)butane-1,2,3,4-tetraol

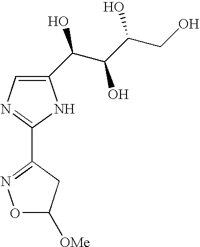

A 1M solution of HCl (10 ml) was added to a room temperature solution of the imidazole 5 (Scheme 3, 500 mg, 1.41 mmol) in MeOH (10 ml). The reaction was heated to 50° C. for 8 h, cooled to room temperature, and concentrated to dryness to provide the title compound (430 mgs, 100% yield) as a slightly yellow powder as a 1:1 mixture of diastereomers. MS m/z C₁₁H₁₇N₃O₆ [M+H]⁺=288; ¹H NMR (400 MHz, D₂O) δ 7.06 (s, 1H), 5.71 (d, J=7.2 Hz) and 5.41 (d, J=7.2 Hz, 1H), 4.72 (s, 1H), 3.2-3.4 (m, 3H), 2.98-2.80 (m, 2H).

### 5.9. Synthesis of (1R,2S,3R)-1-(2-(5-methyl-1H-pyrazol-3-yl)-1H-imidazol-5-yl)butane-1,2,3,4-tetraol

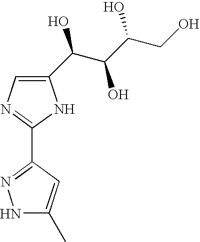

The title compound was prepared from 1-(5-((4S,4′R,5R)-2,2,2′,2′-tetramethyl-4,4′-bi(1,3-dioxolan)-5-yl)-1H-imidazol-2-yl)ethanone (compound 9) as follows. A solution of 9 (975 mg, 3.15 mmol) in THF (15 ml) was added slowly to a −10° C. solution of potassium hexamethyldisilazane (15.72 ml of a 0.5 M toluene solution, 7.86 mmol) in THF (15 ml). The reaction was maintained at −10° C. for 10 min before the addition of ethyl acetate (1.55 ml, 15.75 mmol). The reaction was warmed to room temperature for 1 h, then quenched by the addition of 30 ml NH₄Cl (sat. aq.). The layers were separated, and the aqueous layer was washed with EtOAc (2×30 ml). The combined organics were washed with water (30 ml) and brine (30 ml), then dried over MgSO₄ and concentrated. The resulting tan material was used without further purification.

The crude material was dissolved in EtOH (20 ml) and acidified with 1N HCl (5 ml). The stirred, room temperature solution was then treated with excess hydrazine monohydrate (200 μl). At completion, the reaction was adjusted to pH=7 with 1 N NaOH, then concentrated to a ˜10 ml volume. DCM (30 ml) was added to dissolve the solids which had precipitated from the aqueous solution, and the layers were separated. The organic layer was dried over MgSO₄ and concentrated. The crude was flashed over silica (5-10% MeOH:DCM eluent) to provide the protected pyrazole (204 mg, 19% yield) as a clear foam.

A solution of 1N HCl (5 ml) was added to a room temperature solution of the protected heterobicycle (180 mgs, 0.52 mmol), and the reaction was heated to 50° C. After 1.5 h, the reaction was cooled to room temperature, then concentrated to dryness. The foam was re-dissolved in 2 ml MeOH, then triturated with 3 ml Et₂O and cooled to 0° C. before decanting the liquids. The solid was washed with Et₂O (2×2 ml), then dried under a high vacuum to provide the title compound (130 mgs, 70% yield) as a white powder. MS m/z C₁₆H₁₆N₄O₄ [M+H]⁺=269; ¹H NMR (400 MHz, D₂O) δ 7.28 (s, 1H), 6.52 (s, 1H), 5.07 (d, J=0.9 Hz, 1H), 3.74-3.54 (m, 4H), 2.22 (s, 1H); ¹³C NMR (D₂O): δ 142.8, 139.1, 136.3, 134.1, 116.0, 104.0, 72.6, 70.6, 64.4, 62.7, 9.6.

### 5.10. Measuring Effects on Lymphocytes in Mice

Compounds were administered by oral gavage or in drinking water. For oral dosing experiments, compounds were resuspended from crystals at 10 mg/ml in vehicle (e.g., water). Mice (F1 hybrids of 129/B6 strain) were gavaged with a single 100 mg/kg dose of compound (equivalent to 100 mpk of the free base for each compound) or a vehicle-only control, and returned to their cages. Mice were anesthetized using isofluorane eighteen hours after dosing and tissues were collected for analysis as described below. For drinking water studies, compounds were dissolved at 50 mg/L in acidified water (pH=2.8) containing 10 g/L glucose. The mice were allowed free access to compound-containing water (or glucose solution as a control) for 72 hours. At the end of 72 hours, tissues were collected for analysis.

CBC measurements were obtained as follows. Mice were anesthetized with isofluorane and blood was collected from the retroorbital plexus into EDTA blood collection tubes (Capiject-MQK, Terumo Medical Corp., Elkton, Md.). Automated CBC analysis was performed using a Cell-Dyn 3500 (Abbott Diagnostics, Abbott Park, Ill.) or a HemaVet 850 (Drew Scientific, Inc., Oxford, Conn.) instrument.

Flow cytometry (FACS) measurements were obtained as follows. Twenty five μl whole blood was lysed by hyoptonic shock, washed once in 2 ml FACS wash buffer (FWB: PBS/0.1% BSA/0.1% NaN₃/2 mM EDTA) and stained for 30 minutes at 4° C. in the dark with a combination of fluorochrome-conjugated antibodies diluted in 50 μl FWB. After staining, the cells were washed once with 2 ml FWB and resuspended in 300 μl FWB for acquisition.

Standard procedures for non-sterile removal of spleen and thymus were followed. Organs were dispersed into single-cell suspensions by forcing the tissue through a 70 μm cell strainer (Falcon, Becton Dickinson Labware, Bedford, Mass.). For FACS analysis, RBCs were lysed by hypotonic lysis, washed, and 1×10⁶ cells were incubated with 10 μl anti-CD16/CD32 (Fc Block™, BD-PharMingen, San Diego, Calif.) ( 1/10 dilution in FWB) for 15 minutes at 4° C. The cells were stained with a combination of fluorochrome-conjugated antibodies diluted in 50-100 μl FWB, added directly to the cells in Fc Block, for 30 minutes at 4° C. in the dark. After staining the cells were washed once with 1 ml FWB, and resuspended in 300 μl FWB for acquisition. All antibodies were purchased from BD-PharMingen, San Diego, Calif. unless otherwise specified. Samples were analyzed using a FACSCalibur flow cytometer and CellQuest Pro software (Becton Dickinson Immunocytometry Systems, San Jose, Calif.).

Antibody mixes used for the thymus were: TCRb APC Cy7; CD4 APC; CD8 PerCP; CD69 FITC; and CD62L PE1. Antibody mixes used for spleen and blood were: B220 PerCP; TCRb APC; CD4 APC Cy7; CD8 PE Cy7; CD69 FITC; and CD62L PE.

### 5.11. Measuring Effects on S1P Levels in Mice

Levels of S1P in mouse (F1 hybrids of 129/B6 strain) spleen were measured using an adaptation of the radio-receptor binding assay described in Murata, N., et al., Anal. Biochem. 282:115-120 (2000). This method utilized HEK293F cells overexpressing Edg-1, one of the S1P receptor subtypes, and was based on the competition of labeled S1P with unlabeled S1P in a given sample.

HEK293F cells were transfected with a pEFneo S1P receptor (Edg-1)-expression vector and a G418-resistant cell clone was selected. The Edg-1-expressing HEK293F cells were cultured on 12 multiplates in DMEM containing 5% (v/v) FBS in a humidified air:CO₂ (19:1) atmosphere. Twenty four hours before the experiment, the medium was changed to fresh DMEM (without serum) containing 0.1% (w/v) BSA.

Eighteen hours after the test compound was administered, mice were sacrificed and their spleens were removed and frozen. S1P was obtained from the frozen tissue using known methods. See, e.g., Yatomi, Y., et al., FEBS Lett. 404:173-174 (1997). In particular, 10 mouse spleens in 1 ml ice cold 50 mM phosphate buffer (pH 7.5) containing 1 mM EGTA, 1 mM DTT and Roche complete protease inhibitors were homogenized three times at one minute intervals on ice. The result is centrifuged at 2500 rpm and 4° C. for 10 minutes to remove cell debris. The supernatant was then ultracentrifuged at 45000 rpm and 4° C. in a 70 Ti rotor for 1 hour to pull down the membrane-associated proteins. The supernatant was discarded, and the pellet was resuspended in minimal volume (˜1 ml) of ice cold 50 mM phosphate buffer (pH 7.5) containing 1 mM EGTA, 1 mM DTT and 33% glycerol with Roche complete protease inhibitors present. The total protein concentration was measured using the Bradford assay.

S1P was extracted into chloroform/KCl/NH₄OH (pH˜12), and the upper aqueous phase is kept. It was then extracted in chloroform/methanol/HCl (pH<1), and the lower organic phase was kept and evaporated to provide S1P, which was stored in a freezer until used. Just before the assay, the dried sample was dissolved by sonication in a binder buffer consisting of 20 mM Tris-HCl (pH 7.5), 100 mM NaCl, 15 mM NaF, and 0.4% (w/v) BSA.

The S1P content of a sample was measured by a radioreceptor-binding assay based on a competitive binding of [³³P]S1p with S1P in the sample on Edg-1-expressing cells. Edg-1-expressing HEK293F cells in confluent 12 multiplates were washed twice with the ice-cold binding buffer and then incubated with the same buffer containing 1 nM [³³P]S1P (about 18,00 dpm per well) and increasing doses of authentic S1P or test sample in a final volume of 0.4 ml. The plates were kept on ice for 30 minutes, and the cells were washed twice with the same ice-cold binding buffer to remove unbound ligand. The cells were solubilized with a solution composed of 0.1% SDS, 0.4% NaOH, and 2% Na₂CO₃, and the radioactivity was counted by a liquid scintillation counter. The S1P content in the assay well was estimated by extrapolation from the standard displacement curve. The content of S IP in the initial test sample(s) was calculated by multiplying the value obtained from the standard curve by the recovery efficiency of S1P extraction and the dilution factor.

All references (e.g., patents and patent applications) cited above are incorporated herein by reference in their entireties.

1. A pharmaceutical composition comprising an immunosuppressant and an S1P lyase inhibitor of the formula: 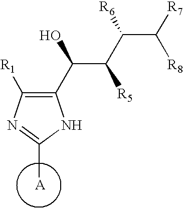
or a pharmaceutically acceptable salt thereof, wherein:
A is an optionally substituted heterocycle;
R₁ is OR_{1A}, OC(O)R_{1A}, C(O)OR_{1A}, hydrogen, halogen, nitrile, or optionally substituted alkyl;
R₅ is OR_{5A}, OC(O)R_{5A}, N(R_{5B})₂, NHC(O)R_{5B}, hydrogen, or halogen;
R₆ is OR_{6A}, OC(O)R_{6A}, N(R_{6B})₂, NHC(O)R_{6B}, hydrogen, or halogen;
R₇ is OR_{7A}, OC(O)R_{7A}, N(R_{7B})₂, NHC(O)R_{7B}, hydrogen, or halogen;
R₈ is CH₂OR_{8A}, CH₂OC(O)R_{8A}, N(R_{8B})₂, NHC(O)R_{8B}, hydrogen, or halogen; and
each of R_{1A}, R_{5A}, R_{6A}, R_{7A}, and R_{8A} is independently hydrogen or optionally substituted alkyl.

2. (canceled)

3. The pharmaceutical composition of claim 1, wherein the S1P lyase inhibitor is of the formula: 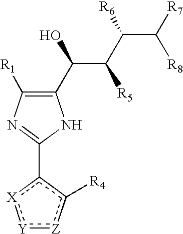
wherein:
X is N or NR₉;
Y is CR₄, N, NR₉, 0 or S;
Z is CR₄, CHR₄, N, NR₉, O or S;
R₁ is hydrogen or optionally substituted lower alkyl; and
each R₄ is independently OR_{4A}, OC(O)R_{4A}, hydrogen, halogen, or optionally substituted alkyl, aryl, alkylaryl, arylalkyl, heteroalkyl, heterocycle, alkylheterocycle, or heterocyclealkyl;
each R₉ is independently hydrogen or optionally substituted alkyl, aryl, alkylaryl, arylalkyl, heteroalkyl, heterocycle, alkylheterocycle, or heterocyclealkyl; and
each R_{4A} is independently hydrogen or optionally substituted alkyl.

4. The pharmaceutical composition of claim 3, wherein X is N.

5. The pharmaceutical composition of claim 3, wherein Y is NR₄.

6. The pharmaceutical composition of claim 5, wherein R₄ is hydrogen.

7. The pharmaceutical composition of claim 5, wherein R₄ is optionally substituted alkyl or alkylaryl.

8. The pharmaceutical composition of claim 3, wherein Y is O.

9. The pharmaceutical composition of claim 3, wherein Z is CHR₄.

10. The pharmaceutical composition of claim 9, wherein R₄ is hydrogen.

11. The pharmaceutical composition of claim 9, wherein R₄ is OR_{4A}.

12. The pharmaceutical composition of claim 9, wherein R_{4A} is lower alkyl.

13. The pharmaceutical composition of claim 3, wherein one or more of R₅, R₆, R₇, and R₈ is hydroxyl or acetate.

14. The pharmaceutical composition of claim 13, wherein all of R₅, R₆, R₇, and R₈ are hydroxyl.

15. The pharmaceutical composition of claim 13, wherein all of R₅, R₆, R₇, and R₈ are acetate.

16. The pharmaceutical composition of claim 1, wherein the S1P lyase inhibitor is:
(1R,2S,3R)-1-(2-(5-methylisoxazol-3-yl)-1H-imidazol-5-yl)butane-1,2,3,4-tetraol;
(1R,2S,3R)-1-(2-(5-ethylisoxazol-3-yl)-1H-imidazol-5-yl)butane-1,2,3,4-tetraol;
(1R,2S,3R)-1-(2-(isoxazol-3-yl)-1H-imidazol-5-yl)butane-1,2,3,4-tetraol;
(1R,2S,3R)-1-(2-(2-methylthiazol-4-yl)-1H-imidazol-4-yl)butane-1,2,3,4-tetraol;
(1R,2S,3R)-1-(2-(5-methoxy-4,5-dihydroisoxazol-3-yl)-1H-imidazol-5-yl)butane-1,2,3,4-tetraol; or
(1R,2S,3R)-1-(2-(5-methyl-1H-pyrazol-3-yl)-1H-imidazol-5-yl)butane-1,2,3,4-tetraol.

17. The pharmaceutical composition of claim 1, wherein the immunosuppressant is aminopterin, azathioprine, cyclosporin A, D-penicillamine, gold, hydroxychloroquine, leflunomide, methotrexate, minocycline, sulfasalazine, or a pharmaceutically acceptable salt thereof.

18. The pharmaceutical composition of claim 17, wherein the immunosuppressant is methotrexate.

19. The pharmaceutical composition of claim 17, wherein the immunosuppressant is cyclosporin A.

20. (canceled)
